(19) United States
(12) Patent Application Publication
(10) Pub. No.: US 2010/0236308 A1
(43) Pub. Date: Sep. 23, 2010

(54) SYSTEM AND METHOD FOR MAKING LIQUID COMPOST

(76) Inventors: Kevin J. Gunn, Carneys Point, NJ (US); Garland T. Swanson, Greenwich, NJ (US)

Correspondence Address: WILLIAM H. EILBERG, 316 CALIFORNIA AVE. #785, RENO, NV, 89509, US

(21) Appl. No.: 12/406,312

(22) Filed: Mar. 18, 2009

(51) Int. Cl. C05F 11/00; C12M 1/10

(52) U.S. Cl. 71/11; 435/290.3

(57) ABSTRACT

Liquid compost is made automatically and continuously. A rotating carousel, mounted above a tank, receives organic material, and a plurality of nozzles spray fluid onto the carousel, so as to entrain particles of organic material. The fluid and the particles pass through a screen in the carousel, and into the tank. Residual organic material which has not passed through the screen is removed from the carousel by a nozzle oriented to push such material off the carousel. A system of conveyors automatically delivers fresh organic material to the carousel, and removes spent organic material from the carousel. The entire system operates continuously, so that liquid compost, or compost tea, can be made efficiently.

### BACKGROUND OF THE INVENTION

This invention relates to the field of making liquid compost from organic material, for agricultural use.

It has been known to produce liquid compost, which is a suspension containing water and composted organic material, such as wood chips and/or animal waste. Liquid compost is typically dispensed through an irrigation system, or through some other spray device, so that the material can be spread over a large area of crops, turf, and/or ornamental plants.

In the prior art, liquid compost has been produced in a batch process. That is, a quantity of organic matter is placed in a vessel, and is then combined with water. The material may be stirred until the desired consistency is reached. The resulting suspension is liquid compost, which is removed from the vessel, at which point another batch can be made. The device used to make the liquid compost is known as an extractor, because the product is extracted from the organic material.

The compost extractors of the prior art are essentially batch processors. While attempts to automate the process have been proposed, the automation has been limited to the use of conveyors to deliver organic material to the extractor. The devices of the prior art cannot be used to produce liquid compost on a continuous basis. Moreover, the task of delivering organic material to the extractor, and removal of spent organic material, is a labor-intensive process which has not been automated in prior art devices. Also, the making of liquid compost is an inherently messy procedure, and creates a disposal problem.

The same problems are encountered in the making of “compost tea”, which is similar to liquid compost, except that the water used to make the product has an oxygen content sufficient to support the growth of microorganisms. A compost tea is typically made by supplying a kit which includes a “tea” bag containing compost, with the addition of other chemicals, and brewing the “tea” in liquid, in the presence of oxygen, for an extended period of time. The resulting compost tea may be used as a fertilizer, or as a composition for preventing plant diseases.

The present invention seeks to improve on prior art processes for making liquid compost or compost tea, by providing a system and method in which liquid compost is produced in a continuous and automatic manner. The present invention automates substantially all of the process, including the loading and unloading of organic material, and the cleaning of the device.

### SUMMARY OF THE INVENTION

The present invention comprises an automated system and method for making liquid compost or compost tea.

In the preferred embodiment, a supply of organic material is brought in a trailer, or its equivalent, to the vicinity of an extraction unit. Organic material from the trailer is automatically conveyed to a hopper located above the extraction unit. The extraction unit includes a carousel which is mounted for rotation above a tank, the tank being stationary. Organic material is dispensed from the hopper, assisted by gravity, onto the rotating carousel. The carousel includes a screen, and a plurality of nozzles capable of spraying water at variable pressures. The liquid compost is formed when organic material is entrained by the water, and the resulting suspension passes through the screen and into the tank below.

The carousel defines a plurality of screen segments. When the carousel rotates, each screen segment is moved through various stations. An inlet station is defined by the hopper, and enables fresh organic material to be deposited onto the screen segment below. Various operating stations follow, in which nozzles direct pressurized water onto the organic material on the screen, thereby producing the liquid compost. Finally, an outlet station includes a horizontally-disposed nozzle and/or scraper which forces spent organic material generally sideways, into a chute, for delivery to a discharge conveyor, so that the spent material can be returned to the trailer. Due to the continuous rotation of the carousel, a segment which has passed through the outlet station next enters the inlet station, and the process automatically repeats.

An air pump directs air into the tank, which stirs its contents, and keeps the consistency of the suspension relatively uniform. Alternatively, the air pump can be used to supply oxygen to the tank to support the manufacture of compost tea, which requires that the liquid remain in the tank for a longer period.

The invention thus automates the process of making liquid compost, by providing a system in which organic material is automatically removed from a trailer, and in which liquid compost is made automatically. Furthermore, that portion of the organic material which does not pass through the screen, and which therefore does not become part of the final product, is automatically returned to the trailer so that it can be carried away.

Moreover, the device is inherently self-cleaning. Simply by stopping the supply of organic material, and by maintaining the flow of water through the nozzles, the system can be substantially flushed without any direct human intervention.

The liquid compost made by the present invention can be sprayed over crops, or over turf or ornamental plants, or used prior to planting, or it can be dispensed through an irrigation system.

The present invention therefore has a primary object of providing a system and method for making liquid compost or compost tea.

The invention has the further object of automating the process of making liquid compost or compost tea.

The invention has the further object of reducing the effective cost of making liquid compost or compost tea.

The invention has the further object of providing an efficient system for making liquid compost or compost tea, wherein the system can be automatically cleaned.

The invention has the further object of providing an improved compost extraction method for providing beneficial organisms, and/or chemical elements contained within the compost used.

The reader skilled in the art will recognize other objects and advantages of the present invention, from a reading of the following brief description of the drawings, the detailed description of the invention, and the appended claims.

### BRIEF DESCRIPTION OF THE DRAWINGS

FIG. 1 provides a side elevational view of the system of the present invention.

FIG. 2 provides a fragmentary perspective view of conveyor apparatus, used in the present invention to transport organic matter from a trailer to a vessel.

FIG. 3 provides a more detailed side elevational view of the vessel used in the present invention.

FIG. 4 provides a top view of the vessel of the present invention, and also showing a portion of an adjacent conveyor.

### DETAILED DESCRIPTION OF THE INVENTION

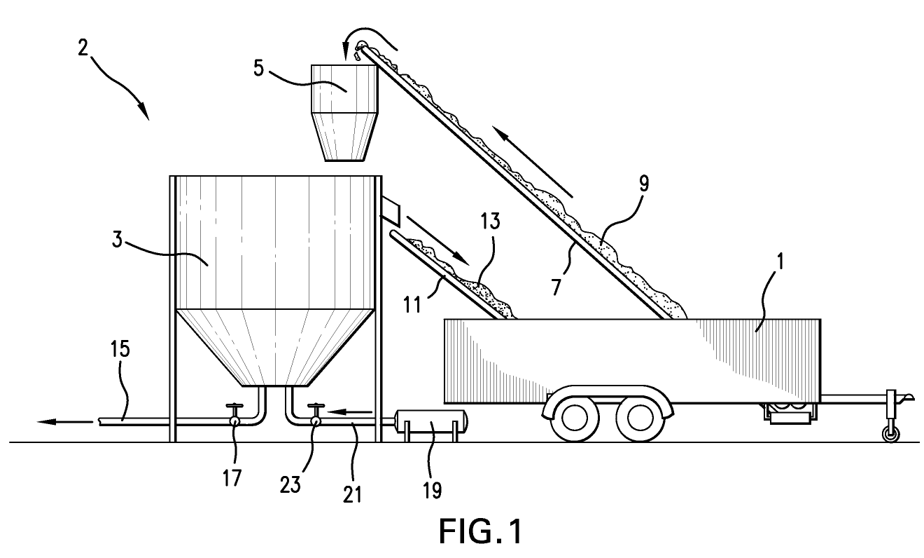

FIG. 1 provides a side elevational view illustrating the present invention in broad outline. A trailer 1 transports organic material to the vicinity of an extraction/brewing unit 2 which comprises vessel 3 and hopper 5. For convenience of description, the extraction/brewing unit will be simply called the extraction unit.

An elevating conveyor 7 transports organic material 9 from the trailer to the hopper. A discharge conveyor 11 transports spent organic material 13 back to the trailer.

Organic material is dispensed from hopper 5 into vessel 3 of extraction unit 2, in a manner which will be described in more detail below. At least some of the organic material is used to make liquid compost, or compost tea, which is eventually withdrawn through conduit 15, as controlled by valve 17. Liquid compost flowing through conduit 15 may be conveyed to an irrigation system (not shown), or to a spray device (not shown), or to any other system which uses liquid compost.

Air pump 19 directs air through conduit 21, as controlled by valve 23, into the vessel. The air from the pump can be used to stir the contents of the vessel, or it can be used to provide oxygen to support the growth of microorganisms in the making of compost tea.

Conduits 15 and 21 may optionally include check valves, to prevent the backflow of product liquid or air, respectively.

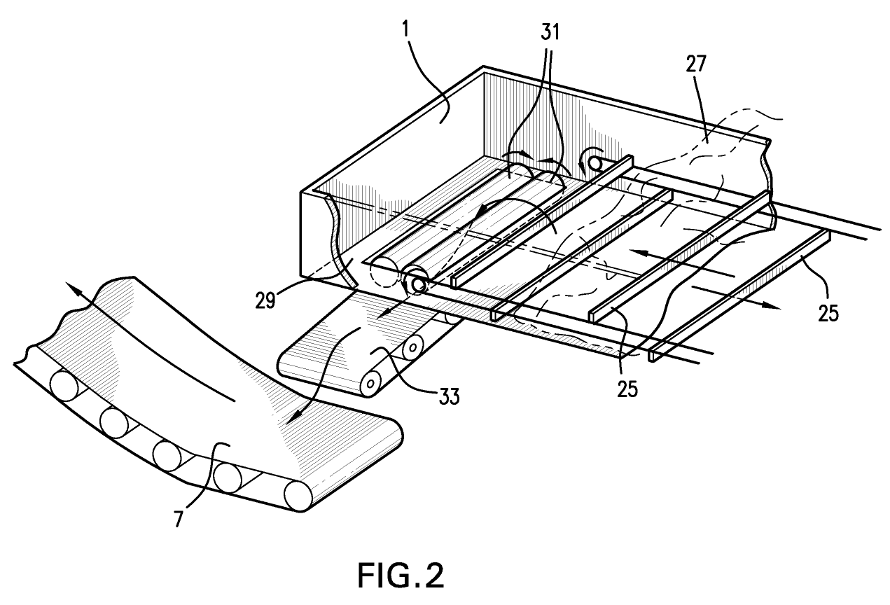

The unloading and re-loading of trailer 1 is preferably performed automatically. The structure of the interior of the trailer is similar to that of a conventional manure spreader. As shown in FIG. 2, the interior of trailer 1 includes a plurality of ribs 25, configured to move together along the length of the flat floor of the trailer. In a preferred embodiment, the ribs are connected to a continuous chain drive. The organic material, indicated in dotted outline by reference numeral 27, is carried by the ribs towards an outlet area 29 of the trailer.

Organic material transported by the ribs passes over rollers 31, which are counterrotating, in a direction such that organic matter tends to be drawn in and pulled between the rollers. Organic material therefore passes between the rollers, and is at least partially crushed, thereby reducing its average particle size. The crushed organic material then falls directly downward, through an opening in the floor of the trailer, onto cross-conveyor 33. The cross-conveyor 33 is positioned such that it carries organic material away from the trailer. The crushed organic material then drops, by gravity, onto elevating conveyor 7, which is the same conveyor shown in FIG. 1.

Thus, organic material is transported within the trailer, removed from the trailer, and carried by conveyor 7 towards hopper 5, all by automatic means.

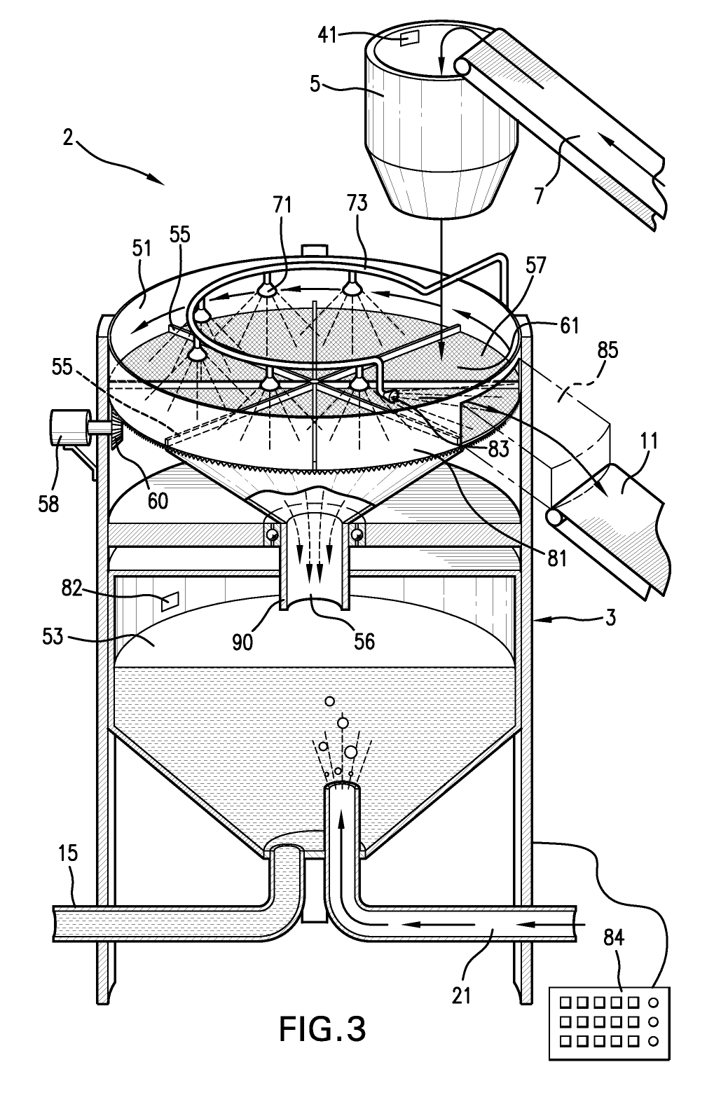

As illustrated in FIG. 3, hopper 5 includes sensor 41 which senses the amount of organic material in the hopper. The output of sensor 41 is preferably used to control the speed of elevating conveyor 7. The speed of elevating conveyor 7 is increased when the hopper is not sufficiently filled, and it is decreased, or even stopped, when the hopper is filled or nearly filled. The sensor 41 can be an electronic device, such as a photoelectric sensor, or a mechanical or electromechanical sensor, or their equivalents.

The conveyors may be moved by any suitable motor, which may be hydraulic, electric, or gasoline-powered. The motors which drive the conveyors preferably have adjustable speeds, so that the speed of the conveyors can thereby be controlled. The motors are preferably designed so that they can be controlled, at least in part, by the output of sensor 41.

The hopper preferably includes a metering means (not shown) which controls the flow of organic material out of the hopper. The metering means may include means for sensing the weight of organic material on the bottom of the hopper, and means for opening the hopper to release such material at an appropriate time. Other metering means could be used instead of that described above, within the scope of the invention.

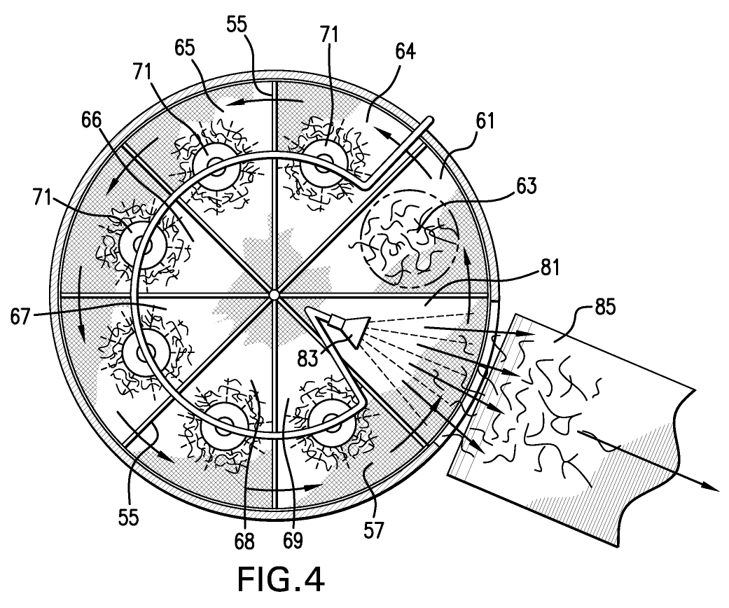

As shown in FIGS. 3 and 4, vessel 3 includes carousel 51 mounted above tank 53. The carousel has the form of a large funnel, which allows material within the carousel to flow downward by gravity. In the embodiment illustrated, the carousel wall is conical. In other embodiments, not shown, the wall of the carousel could have other forms of curvature. For example, the wall could be a domed structure. The invention is not limited to a particular shape; what is important is that material in the carousel be allowed to fall into the tank below.

A motor 58 causes the carousel to rotate. In FIG. 3, the motor is shown coupled to the carousel through gear 60, but other means of coupling could be used instead. The carousel therefore rotates with respect to the tank, preferably at a rate of about 1-3 rpm, the latter speed being exemplary and not limiting.

The motor can be electric or hydraulic, or it could have some other construction. The speed of the motor is preferably adjustable, so that the rotation rate of the carousel can be adjusted. There may also be more than one motor, and/or more than one coupling between one or more motors and the carousel. It is also possible to set the rotation rate at a predetermined level, and to keep that rate constant.

The carousel may include a plurality of dividers 55 which sit atop a fine-mesh screen 57. The screen preferably has a mesh size of about 300-400, but other mesh sizes could be chosen. In the embodiment illustrated, the dividers define eight screen segments of substantially equal size. In practice, the system of the invention can be operated with different numbers of dividers and segments.

In the embodiment shown, the apparatus defines eight stations, which are fixed relative to the vessel 3. Of these eight stations, one comprises an inlet station, another comprises an outlet station, and the other stations comprise operating stations. More particularly, the region immediately below the hopper 5 comprises the inlet station. Organic material, such as material 63, is deposited on screen 57 at this station.

The regions immediately below the nozzles 71 comprise the operating stations. The nozzles are connected by spray bar 73. The spray bar is connected to a source (not shown) of pressurized water, for delivery to the nozzles. In the example shown, each operating station has only one nozzle. But in general, there may be more than one nozzle associated with some or all of the operating stations.

The number of nozzles per station depends, in part, on the size of the carousel. If the carousel is relatively large, it becomes necessary to provide more nozzles, to achieve proper coverage of the area included in each station. For a carousel that is sufficiently small, one nozzle per station may be adequate.

The nozzles 71 deliver water which is preferably at a pressure of the order of about 30 psi. The nozzles 71 are directed downward, so that organic material on the screen is entrained by the water so as to form a suspension. The nozzles are preferably chosen according to the desired flow of water, and the desired operating pressure. At least some of the suspension formed by the organic material and the water passes through the fine-mesh screen 57, through conduit 56, and into tank 53. This suspension comprises liquid compost. Note that the conduit 56 is defined by hollow shaft 90 which serves both to provide mechanical support for the carousel, and to define an exit conduit for the product liquid. The shaft 90 thus comprises a means for guiding fluid from the carousel to the tank.

The tank preferably has a conically shaped bottom, as shown in FIG. 3. In one embodiment, the carousel is approximately 48 inches in diameter, and the tank is capable of holding about 750 gallons. The dividers may have a height of about 1.5-2 inches. All of these figures are given by way of example, and should not be deemed to limit the scope of the invention. The diameter of the carousel, for example, could range from a few inches to virtually any diameter desired. In general, the system of the invention can be made larger or smaller, as needed.

The region in the vicinity of nozzle 83 comprises the outlet station. Unlike the other nozzles, nozzle 83 is oriented generally horizontally, i.e. parallel, or approximately parallel, to the surface of the screen. The pressurized water thereby pushes residual organic material from screen segment 81 into chute 85, which allows the material to fall, by gravity, onto discharge conveyor 11.

Thus, the rotating carousel brings the various segments 61, 64, 65, 66, 67, 68, 69 into registration with the various stations. The carousel rotates, but the nozzles and the hopper do not. Thus, each segment changes position, and moves from one station to another.

More particularly, a given screen segment starts at the inlet station, where organic material is deposited onto the segment. Since the carousel rotates in a counterclockwise direction, as viewed from above the carousel (and as indicated by the arrows in FIGS. 3 and 4), the given segment then passes through the various operating stations, where the nozzles 71 direct water onto the screen so as to entrain the organic material, and thereby produce liquid compost. Finally, the segment arrives at the outlet station, where nozzle 83 forces spent organic material into the chute, to be returned to the trailer.

The segment will proceed next to the inlet station, and the above process repeats itself. In general, the hopper can be depositing organic material continually, or almost continually, onto the segments of the screen as they pass by the hopper.

The extraction unit 2 therefore produces an extract from the organic matter, through a continuous washing process. Organic material is automatically and continuously loaded onto the carousel, and is continuously washed with high-pressure water, so as to produce liquid compost. Residual organic material is automatically removed from the carousel and returned to the trailer.

Instead of using water to remove residual organic material from the carousel, one could use a stream of air, or a stream which includes both water and air. A mechanical device (not shown) could also be added for assisting in the removal of the residual organic material.

As described above, the combination of the trailer with the extraction/brewing unit is especially advantageous, because organic matter can be automatically unloaded from the trailer, processed in the extraction unit, and residual organic matter can be automatically returned to the trailer for disposal. However, it is possible to use the extraction/brewing unit alone, without the trailer, in which case other means of supplying and removing organic matter must be provided. It is also possible to replace the trailer with some other structure. Furthermore, the residual organic material may be conveyed to a holding device other than the trailer.

The embodiment illustrated in the drawings includes only one carousel. It is also possible, within the scope of the invention, to provide multiple carousels, which could be stacked vertically, separated by appropriate spacers. Such an arrangement would enable the apparatus to process more organic material in a given time.

The tank 3 can be used both for simply holding liquid compost which has just been made, or for brewing a compost tea. The air supplied through conduit 21 can thus be used to agitate the contents of the tank, to keep the organic material in suspension, or to provide oxygen to the contents of the tank, to promote brewing. If one seeks to make compost tea, the contents of the tank must remain there for an extended period of time. In the case of a compost tea, it may be necessary to provide a means for supporting a compost tea kit, which would include compositions for supporting the desired growth of microorganisms.

The vessel 3 is preferably equipped with means (not shown) for starting and stopping the process of filling the tank. Such means may include means for monitoring the liquid level in the tank. Such monitoring may be done by sensor 82, which could be a photoelectric sensor, or a mechanical arm, or some other sensing device. The output of sensor 82 can be connected to the source of water, and can stop the flow of water if the tank is full.

Liquid compost, or compost tea, produced by the extractor 2, can be pumped into another tank which can be used for transporting, spraying, or storage of the product. The product can also be conveyed to an irrigation system for dispersal through that system. The apparatus may include a means (not shown) for varying the rate at which liquid is dispensed into the irrigation system, and for measuring the amount of liquid supplied.

The system of the invention may also include means (not shown) for connecting the extraction unit directly to an irrigation system.

The system of the invention may be provided with space for the storage and use of additional products which can be automatically added or metered into the brewing and/or extraction process.

The trailer is preferably equipped with a lifting tailgate, to facilitate unloading of spent organic material which has been returned to the trailer.

The extraction unit preferably is computer-controlled, and includes control panel 84 for directing the operation of the unit. In the preferred embodiment, the device is capable of starting automatically in response to the start of water flow in a connected irrigation system (not shown). When the water flow in the irrigation system stops, the device can be programmed to clean itself and then automatically shut down. The control panel preferably also includes means for controlling the extraction unit manually. Among other parameters to be controlled, the control panel can allow the user to set the conveyor speeds and the compost flow rates. The control panel could also control the inflow of other products which may be injected into the tank, especially in the case of the brewing of compost tea.

In the preferred embodiment, the extraction device is also provided with various safety features, including the ability to accommodate power failures or overflows. The device is also capable of metering specific preselected amounts of liquid compost.

The operation of the system of the present invention is summarized as follows. A trailer, loaded with organic material, is positioned adjacent to the extraction unit. An elevating conveyor is positioned over the feed hopper of the extraction unit. Hydraulic or electrical connections are established between the trailer and the extraction unit. The organic material is delivered automatically from the trailer, through the use of a continuous chain drive in the trailer, and through the use of a cross-conveyor, to the elevating conveyor, so that the crushed organic material enters the hopper.

The hopper evenly dispenses organic material onto the screen contained in the rotating carousel of the extraction unit. As the carousel turns, water is sprayed through the organic material, causing much of the organic material to be entrained by the water, and to pass through the screen, thus forming a suspension comprising liquid compost. When the carousel has made a full rotation, the residual organic material, which has not passed through the screen, is washed off the screen, and deposited onto the discharge conveyor, which leads downward to a position in the trailer which is at the opposite end from the section where organic material was removed. The spent compost falls off the discharge conveyor, and is thus automatically loaded into the trailer.

When the fresh organic material delivered by the trailer has been used up, and the spent organic material has been loaded in the trailer, the trailer is transported to the original compost pile, and emptied and reloaded. The process can then be repeated, as needed.

Since the present invention may conveniently be used in conjunction with an irrigation system, the source of water, for the device of the present invention, is preferably the same source used for the irrigation system. Any excess waste and/or water can be returned to the source of irrigation water.

The speeds of the conveyors are preferably adjusted and controlled by computer, preferably with a touch pad or touch screen interface, or otherwise by manual means. The same controls can determine the water pressure and flow rate of water through the system. The computer can also monitor amounts of organic material being processed.

The invention is inherently self-cleaning, because of its use of spray nozzles and high-pressure water. By cutting off the supply of organic material, and by causing the water to flow, one can clean substantially the entire machine automatically.

The extraction unit of the present invention can be connected to the irrigation system so as to allow the automatic transfer of water between the systems. The invention can conveniently be used with different kinds of systems, as it is relatively self-contained.

The present invention has the further advantage that the same device can be used to make either liquid compost or compost tea.

The invention can be modified in various ways. As indicated above, the size of the extraction unit, and its components, can be varied, as the system can be scaled up or down as required. The number of segments of the carousel can be changed, as can the number and position of the various nozzles. The structure of the screen could be varied; other structures which perform the equivalent of the function performed by the screen, could be substituted.

The structure of shaft 90 could be modified. Instead of having the structure shown in the drawings, the shaft could extend from a position below the carousel, to a position above the carousel. The shaft could become the means for rotating the carousel, instead of motor 58 and gear 60.

Another modification of the invention could be the omission or reconfiguration of the tank 53. In the more general case, it is necessary only to have some form of fluid collection means, positioned below the carousel, to collect fluid and organic material which passes through the screen. In the preferred embodiment, shown in the drawings, the tank 53 comprises the fluid collection means. The use of the tank enables a single apparatus to be used for making either liquid compost or compost tea. But the tank could be omitted, and replaced simply by a conduit which could be connected directly to an irrigation system or to some other storage or dispensing system. In the latter case, the conduit would comprise the collection means.

In still another modification of the device of the invention, one could omit the dividers provided on the screen of the carousel. Instead, the screen could comprise one continuous segment. The various positions along the screen would still travel from one station to the next. Also, a mechanical scraper (not shown) could be used in addition to, or instead of, the horizontally-disposed nozzle 83, for removal of residual organic material from the carousel.

The above-described modifications, and others which should be apparent to those skilled in the art, should be considered within the spirit and scope of the following claims.

What is claimed is:

1. An automated system for making liquid compost, comprising:
a) a carousel, the carousel being mounted for rotation above a fluid collection means, the carousel including a screen,
b) means for conveying organic material to a region above the carousel, and means for depositing said organic material onto the carousel,
c) means for directing liquid onto the screen so as to form a suspension containing some of the organic material and the liquid, wherein the suspension can flow through the screen and into the collection means,
d) means for forcing spent organic material off the screen and into a discharge means for removing said spent organic material from the carousel.

2. The system of claim 1, wherein the collection means comprises a tank located below the carousel.

3. The system of claim 1, wherein the conveying means includes a trailer for storing organic material, and means for moving said organic material onto an elevating conveyor, the elevating conveyor having an upper end which is positioned above the carousel.

4. The system of claim 1, wherein the depositing means comprises a hopper.

5. The system of claim 1, wherein the directing means comprises at least one nozzle, the nozzle being connected to a source of pressurized liquid, the nozzle being pointed generally towards the screen.

6. The system of claim 1, wherein the forcing means comprises a nozzle which is oriented so as to urge organic material on the screen away from the screen.

7. The system of claim 1, wherein the discharge means includes a chute for receiving spent organic material, the chute being positioned to direct spent organic material onto a discharge conveyor.

8. The system of claim 2, further comprising an air pump connected to direct air into the tank.

9. A system for making liquid compost, comprising:
a) a vessel which includes a fluid collection means, and a carousel mounted for rotation above said fluid collection means, the carousel including a screen, the carousel having means for guiding fluid from the carousel to said fluid collection means, and
b) means for directing pressurized water onto the carousel such that at least some of the water passes through the screen and into the fluid collection means.

10. The system of claim 9, wherein the fluid collection means comprises a tank positioned below the carousel, and wherein the directing means is fixed relative to the tank.

11. The system of claim 9, further comprising means for spraying pressurized water in a direction such that organic material located on the screen is pushed off the screen.

12. The system of claim 9, further comprising a hopper located above the carousel, the hopper comprising means for depositing organic material onto the carousel.

13. The system of claim 9, wherein the directing means and the spraying means comprise nozzles connected to a spray bar.

14. The system of claim 10, further comprising an air pump for directing air into the tank.

15. The system of claim 14, further comprising a conduit for withdrawing contents of the tank.

16. A method for automated manufacture of liquid compost, the method comprising:
a) depositing organic material on a carousel which is located above a fluid collection means and mounted to rotate relative to the fluid collection means, the carousel having a screen, and
b) directing pressurized fluid towards the organic material so as to entrain some of the organic material and to push some of the organic material through the screen and into the fluid collection means, wherein fluid and organic material accumulate in the fluid collection means to form liquid compost,
wherein steps (a) and (b) are performed while rotating the carousel relative to the fluid collection means.

17. The method of claim 16, further comprising spraying pressurized fluid towards spent organic material which remains above the screen, the spraying step being performed so as to remove said spent organic material from the carousel.

18. The method of claim 16, wherein the fluid collection means is selected to be a tank, and wherein the method further comprises directing air into the tank so as to agitate liquid compost in the tank.

19. The method of claim 16, further comprising automatically delivering fresh organic material to the carousel, and automatically removing spent organic material from the carousel, the delivering and removing steps being performed continuously.

20. The method of claim 16, wherein the fluid collection means is selected to be a tank, and wherein the method further comprises automatically cleaning the carousel and tank by stopping a flow of fresh organic material into the carousel, and spraying pressurized fluid through the directing means, so as to flush contents of the carousel and the tank.
